(19) United States
(12) Patent Application Publication
(10) Pub. No.: US 2006/0172996 A1
(43) Pub. Date: Aug. 3, 2006

(54) Method of preventing relapse in the abstinent substance dependent individual

(76) Inventor: James A. Halikas, Napels, FL (US)

Correspondence Address: James A. Halikas, 783 Tramore Lane, Naples, FL, 34108, US

(21) Appl. No.: 11/126,742

(22) Filed: May 12, 2005

Related U.S. Application Data
(63) Continuation-in-part of application No. 11/049,067, filed on Feb. 3, 2005.

(51) Int. Cl. A61K 31/551

(52) U.S. Cl. 514/220

(57) ABSTRACT

A method of preventing a relapse in the abstinent substance dependent person. The method involves the use of the medication Flumazenil in a spray delivery device and to teach the person to self-administer the medication by spraying the medication into either the nasal passage, the buccal mucosol membranes or the pulmonary membranes, whenever the need and the urge to relapse arises, thereby preventing relapse.

### RELATED APPLICATIONS

This application is a Continuation-In-Part of application Ser. No. 11/049,067

### BACKGROUND OF THE INVENTION

Alcohol and substance dependence are considered to be among the top three disorders in the United States and are associated with serious medical and economical consequences. While alcohol has been recognized as a disease for some time, only recently have we begun to realize that it fits into the same pathways as other addictions. Treatment strategies for the most part have been psychological in nature although pharmacological options are now available in combination with psychological interventions with some additional success. Recently, there have been studies focusing on the reduction of drinking vs. abstinence as an end point. There have also been new issues raised as to predictors of outcomes and adherence to the treatment strategies. These issues along with new technologies and other pharmacological options for the treatment of alcohol and other substance use dependency are part of the innovations of this application.

Psychosocial therapies, despite some demonstrated efficacy, are of limited benefit for a substantial proportion of substance abusers in the different phases of their illness. Medications can augment the effects of psychosocial therapies, increasing the proportion of patients who may respond well to treatment. For example, Disulfiram, the deterrent medication that was approved more than 50 years ago for the treatment of alcoholism, has not consistently been shown to be efficacious. However, it does work in the well motivated patient as an assist to curb impulse use. Recent interest in medications to treat alcoholism has yielded a number of promising candidates. In 1994, Naltrexone, an opiate antagonist, was approved by the FDA. Its success requires a program of education that addresses the reluctance of many treatment providers to consider medications as a key ingredient in alcoholism treatment. Naltrexone, for example, has not been widely prescribed. Variable findings of efficacy, possibly due to limited compliance with oral Naltrexone therapy, appear to limit the drug's clinical acceptability. Recently a long-acting formulation of Naltrexone, not yet released, has been found to enhance the beneficial effects of a psychosocial intervention in the treatment of alcohol dependence. If this monthly injection is accepted it may represent a useful addition to the therapeutic armamentarium for alcoholism.

Treatment attrition and non-adherence to programmatic regimens are major barriers to an adequate response to effective treatments. As is well recognized, treating substance dependent patients is complex, as they frequently fail to follow through with prescribed treatments. It is also well documented that substance dependent patients irregularly attend clinic visits and many discontinue treatment prematurely. As the FDA approves pharmacological therapies for use in the rehabilitative phase of the treatment of alcohol dependence, treatment adherence may be further compromised when patients skip medication doses, or fail to return for prescription renewals—as evidenced in studies of Naltrexone where fewer than 61% of alcohol dependent patients consistently took their oral dose of medication over a twelve-week course of treatment.

This negative impact of patient non-adherence to treatment on outcomes, i.e., not showing up at treatment sessions, skipping medication dosages, etc. is clear. Predictors of non-adherence and new approaches to monitoring non-adherence are part of the treatment protocols now intervention being designed by the Government. Most recent work targets the development of both pharmacological technology and psychosocial interventions that compel medication adherence and treatment retention in patients with alcohol dependence.

Alternatively, the inventive concept in this application uses an entirely different strategy by putting control back in the patients' hands, and by building on their own motivation to maintain abstinence. This new treatment technology avoids all coercion by being voluntary, spontaneous, truly at the will of the patient, and provides almost instantaneous calming of any inner disquiet, inner tremors or inner shakiness.

This invention takes advantage of several aspects.

It is known that drug dependency is a multistage disease, having numerous medical, psychological, psychosocial, and treatment phases. These phases include, for example, such obvious ones as acute intoxication, delirium, detoxification, withdrawal, post withdrawal syndrome, early recovery phase, etc.

After a patient undergoes initial detoxification from, for example, an acute alcohol intoxication, there is always a tendency for a patient to relapse. This is quite an issue because the patient with, for example, an alcohol dependency is a different person when compared to a normal person who is not dependent on alcohol or other substances. The normal person can drink or not drink, that is, socially, without the crucial loss of control and adverse consequences. The patient with alcohol dependency continues to drink in spite of adverse consequences, such as drunk driving, getting fired from employment, domestic altercations, change in personality and memory blackout that may occur. These alcohol related events are not understood but their occurrence appears to include a genetic element for most people. It is not so much an overdose of the substance as much as a poorly understood consequence of its effect in the brain in the susceptible individual.

There are effective pharmacological treatments for alcohol detoxification and detoxification of the rest of the sedative hypnotics, but the pharmacological therapy for prevention of a relapse of the patient is not well understood or treated. There is one set of medications that deals with the craving of alcohol, believing that if one diminishes the craving, one can reduce the chance for relapse. Two such medications are known as Naltrexone and Acamprosate. There are deterrents such as Antabuse, Disulfiram. If this deterrent is taken in the morning and one drinks in the afternoon, one gets deadly sick and that is believed to extinguish one's wish to drink because of the fear of getting sick.

Then there are the concepts that a person perhaps experiences a relapse because of being the subject of depression, anxiety or other psychiatric issues. A prophylaxis for this event would be to administer antidepressants and anti anxiety medications.

The phenomenon of a relapse in a dependent individual is still not understood. A relapse is associated with an ongoing abstinent state long after completion of a physiological withdrawal from any metabolic, addictive or dependent conditions. This state of relapse vulnerability is as yet poorly characterized physiologically, metabolically, biochemically, or neurochemically, for each of the drug or alcohol conditions subsumed in this patent application. This state of vulnerability is nevertheless clearly evident to all families and caregivers of such patients for months and months after detoxification and “rehabilitation” treatment for substance abuse. The relapsing nature of these disorders and the lack of any effective treatment for this phase of the illness are all too apparent to society.

The above analysis of phases of substance abuse, uses alcoholism as an example because it is most prevalent. Alcohol is easily available and at a relative low cost. Alcohol is socially acceptable as a social lubricant as long as it is consumed in moderation. However, the problem is, that a large portion of the population abuses the use of alcohol resulting in adverse effects described above.

There are many medications or substances which are abused or to which individuals become dependent resulting in the same or similar adverse consequences and similar cycles as were noted above. Such substances are Benzodiazepines, including Librium, Valium, Xanax and Ativan, Marijuana, other sedative hypnotics such as Barbiturates, Doriden, Miltown, Soma, Quaaludes, Baclofen, GHB, Neurontin or other GABA active drugs. This application and treatment will be applicable to dependency conditions involving any of these substances or categories of substances.

### BRIEF DESCRIPTION OF THE INVENTION

The medication Flumazenil has been in the medical literature for at least 15 to 20 years. It is a Benzodiazepine antagonist, a receptor blocker that blocks the action of Librium, Valium, Xanax, Ativan, or other Benzodiazepines These medications are addictive and create a dependency in themselves. It turns out that there is a receptor in the brain for these molecules, the Benzodiazepine receptor.

Flumazenil blocks the Benzodiazepine receptor, or, in the alternative, if one has taken one of these medications (one of the Benzodiazepines) and if Flumazenil is given to a patient, it will push out these molecules, and will place the patient into an instant withdrawal state. Thus, Flumazenil is routinely used in an emergency room setting when people have overdosed with Benzodiazepines and almost kill themselves.

Intravenous Benzodiazepines are used in an operating room as rapid anesthetics to put people to sleep and in a reverse action Flumazenil is used to wake them up.

Flumazenil is a medicine also used to reverse the effects of Benzodiazepines (e.g. diazepam and temazepam) which are often used to induce sedation prior to minor outpatient procedures, such as colonoscopies.

The Benzodiazepines work by acting on receptors in the brain (GABA receptors) causing the release of a chemical called GABA (gamma amino butyric acid). GABA is a major inhibitory chemical in the brain involved in inducing sleep and control of trembling. Benzodiazepines act by increasing the activity of GABA, thereby reducing the functioning of, certain areas of the brain. This results in sleepiness, a decrease in shaking and relaxation of muscles.

Flumazenil reverses the effects of Benzodiazepines by competing with them at the GABA receptors. Flumazenil binds to the receptors, preventing Benzodiazepines from acting on them. This blocks their effect and causes sedation to be reversed.

This invention uses this medication “Flumazenil” for a new use in several surprising new methodologies, that is, in various types of convenient spray delivery devices and direct absorption systems, never before used for this medication, and for a stage of illness of this group of diseases never before dreamed of, under the unheard of control of the patient, with excellent clinical results.

To prevent the relapse to the urges of a substance under the control of a patient through a novel treatment mechanism via a novel new route is remarkable.

### DETAILED DESCRIPTION OF THE INVENTION

Human use has found that if the substance Flumazenil is transferred and placed into the blood stream of a person as quickly and as efficiently as possible during a period of urges for a substance of abuse, such as alcohol, in an otherwise abstinent person, the urge will disappear. While an IV might accomplish the above noted task, it is not realistic for a person, after having undergone detoxification, to have repeated IV therapies daily, to prevent a relapse so as not become substance abuse dependent again. Such a person would have to go to a treatment location where an IV can be administered.

The quickest and most effective way is to place the substance (Flumazenil) into spray devices so that the patient can administer the substance (Flumazenil) to him- or herself. For example, the blood vein endings in the nasal passages, in the buccal mucosal membranes and in the tissues of the lungs are numerous and are very sensitive and readily and quickly absorb the medication into the blood stream. The very rapid absorption into the blood stream results in the by-pass of the liver and the intestines, avoiding the so-called “first pass effect” where so much medication is destroyed. The advantage of such a treatment is that the person can carry any of the spray devices on his or her person and instantly use the spray when such person feels uncomfortable or feels the urge for a use of the substance. The sprays are used on a PRN basis (as necessary basis). Spontaneous control is how a normal user of alcohol controls his/her intake of alcohol. A patient with the disease of substance dependency does not have that spontaneous control. These technologies and this invention allows the patients to train themselves and learn self-control and maintenance of abstinence spontaneously.

The key element in this inventive concept is a series of delivery systems by way of the above identified sprays and using any one of them to prevent a relapse. This treatment is may overcome immediate anxiety, or depression but its primary purpose is just to prevent a relapse into the use of substances of abuse such as alcohol or other sedative hypnotics.

The manipulative steps for the patient is to carry any of the spray devices around wherever the person goes. The patient has to learn to recognize the situations of high relapse or the danger of a high likelihood of a relapse. The patient can thus learn and modify any approaching high risk of a relapse. This is called “relapse prevention techniques and coping skills” by the field. This retraining of spontaneous control and the prevention of relapse is similar in all of the drug or substance abuse dependencies encompassed within this inventive concept.

### Example of Treatment—Case Report

E.G is a 59 yr. old Male with alcoholic cirrhosis, drinking abusively for more than 30 years, who had previously completed four rehabilitation treatment programs, and attended AA meetings on numerous occasions. He was hospitalized during an alcoholic bender with elevated liver enzymes, SGPT 85, SGOT 413, alk phos 352, ALB 2.5, T.BILI. 5.4, ammonia 99, EKG with prolonged QT, marcocryptic anemia and low platelet count. He was detoxified as an inpatient for seven days and then discharged to an outpatient pschosocial group treatment.

At his first outpatient follow up visit, he complained of awakening with panicky feelings and shaky hands. He also claimed dizziness, vertigo, and a variety of somatic complaints. He had not relapsed into alcohol use. He was started on the inventive Flumazenil nasal spray under his own control to use as prn medication.

He returned five days later to say that the spray worked well. He used it as needed, mostly at night. His mood was better; he was less anxious; his health felt subjectively better; it had been an easier few days; the groups seemed to be going well. During the next ten days he continued to use the spray on as needed basis, about once a day. He felt that it was not sedating him; that it seemed to calm him; that it might be improving his mood; he was not thinking about alcohol during treatment discussions about it in group meetings. He did not feel that it was in any way altering his consciousness or making him “high or stoned.”

At the next visit, he noted that he had been in situations with family and friends where alcohol had been available and “I don't want it anymore.” He felt the inventive spray continued to be useful. He was pleased with himself.

On several subsequent visits he continued to do well, sometimes using the spray as often as 2-3 times per day, sometimes skipping days. His mood was good and he had few thoughts of alcohol, his thinking was clear, he felt content, he was in a good frame of mind and he continued to attend group meetings. As time moved on, his personal situation improved because, he believed, of his sobriety and mood stability. He had rare urges for alcohol which passed with breathing exercises and the occasional use of the spray. He was now able to use relaxation techniques and what he had been learning in group meetings as well. His mood continued to be good. He felt that the occasional use of the sprayer helped relieve tension and pressure to lead to urges unexpectedly. Abstinence was being maintained at 90 days, his longest sobriety in many years.

The above example of a treatment of an alcoholic dependent person is just one example of the inventive treatment because of the wide spread abuse of alcohol. However, it is equally applicable in the treatment of persons that are suffering under most all other substance abuses as were enumerated above. The results will be similar or the same as were described above in the example.

The above example of a treatment of an alcoholic dependent person using a nasal spray is only one example.

It has been found that other treatment technologies are equally effective. A second technology is the spray application into a mouth of a person whereby there is an immediate absorption of the medication into the buccal mucosal membrane in the mouth of a person.

A third treatment technology is a pulmonary inhaler earosolization whereby the administered spray will enter the tissues of the lungs directly.

In all of the above treatment technologies it is most important that the medicated sprays enter the blood stream as quickly as possible to be most effective.

It is substantially immaterial which one of the three technologies should be used or are recommended for any given situation or person. It is a matter of preference or tolerance as long as the medication is entering the blood stream as quickly as possible by whatever technology selected.

The inventive concept is not only applicable to prevent a relapse in the abstinent alcohol dependent person but has been found that all the other substances disclosed herein can equally be treated in the same way while achieving the same results.

What I claim is:

1. A method for treating a substance abuse relapse potential or likelihood in a substance abuse human while being in an abstinence mode, said method comprising the step of administering to said human an effective therapeutic amount of a medication, said administering is undertaken via a medicated spray intake into the mouth passage of said human, on as needed basis, when said human feels the immediate subjective premonition or danger of resorting back to the use of an abusive substance.

2. The method of claim 1, wherein said step of administering is a self-administering step.

3. The method of claim 1, wherein this is a wholly new use of said medication named Flumazenil.

4. The method of claim 1, wherein the step of administering consists of a buccal mucosal membrane spraying.

5. The method of claim 1, wherein the step of administering consists of a pulmonary aerosolization inhaler.

6. The method of claim 1, wherein said abusive substance is selected from the group consisting of: Alcohol, Benzodiazepine, Librium, Xanax, Ativan, Marijuana, Doriden, Miltown, Soma, Quaaludes, Baclofen, Neurontin, Ambien, GABA active drugs, GHB, and sedative hypnotics.
